(19) United States
(12) Patent Application Publication
(10) Pub. No.: US 2022/0388973 A1
(43) Pub. Date: Dec. 8, 2022

(54) PROCESS TO CONTINUOUSLY PREPARE A CYCLIC CARBONATE

(71) Applicant: NEW GREEN WORLD B.V., Gouda (NL)

(72) Inventors: Frank VERGUNST, Gouda (NL); Sander LAURENTIUS, Capelle aan den IJssel (NL)

(73) Assignee: NEW GREEN WORLD B.V., Gouda (NL)

(21) Appl. No.: 17/771,223

(22) PCT Filed: Nov. 12, 2020
(86) PCT No.: PCT/EP2020/081897

§ 371 Date: Apr. 22, 2022

(30) Foreign Application Priority Data
Nov. 15, 2019 (NL) 2024243
May 22, 2020 (NL) 2025647

(51) Int. Cl. C07D 317/36

(52) U.S. Cl. CPC C07D 317/36

(57) ABSTRACT

The invention is directed to a process to continuously prepare a cyclic carbonate product by reacting an epoxide with carbon dioxide in the presence of a heterogeneous catalyst activated by an activating compound. The process is performed in a first, second, third reactor, each reactor comprising a slurry of the heterogeneous catalyst and the cyclic carbonate product as present as a liquid. To the first reactor carbon dioxide and the epoxide compound is continuously supplied, liquid cyclic carbonate is discharged and unreacted carbon dioxide and epoxide is discharged as a first gaseous effluent stream to the second reactor while substantially all of the heterogeneous catalyst remains in the first reactor. To the third reactor the activating compound is added. In a next step of the process the third reactor becomes the second reactor, the second reactor becomes the first reactor and the first reactor becomes the third reactor.

Process to continuously prepare a cyclic carbonate product by reacting an epoxide compound with carbon dioxide in the presence of a heterogeneous catalyst which catalyst is activated by an activating compound and wherein the process is performed in at least a first, second, third reactor, each reactor comprising a slurry of the supported catalyst and the cyclic carbonate product as present as a liquid.

US2017197931 describes a process to prepare ethylene carbonate by reacting carbon dioxide with an ethylene oxide in three fixed bed adiabatic reactors in series at 8 MPa and reactor temperatures of between 60 and 140° C. Bromo-ethanol is added with the feed to activate the catalyst.

EP2257559B1 describes a continuous process to prepare ethylene carbonate from ethylene oxide and carbon dioxide is described. The reaction takes place in the presence of a dimeric aluminium salen complex supported on a modified SiO₂ support as the catalyst and nitrogen gas. The supported catalyst is present in a tubular reactor and the reactants are supplied to the tubular reactor as a gaseous mixture of ethylene oxide, carbon dioxide and nitrogen. The temperature in the reactor was kept at 60° C. by means of a water bath and the pressure was atmospheric. The yield of ethylene carbonate was 80%.

An advantage of the process of EP2257559B1 compared to for example the above referred to US2017197931 is that the reaction conditions may be close to ambient in terms of temperature and pressure. As a result of this the energy consumption of the process is low and less by-products are formed. A disadvantage however of the continuous process described in EP2257559B1 is that the tubular reactor requires external cooling to avoid overheating as a result of the exothermal reaction to ethylene carbonate.

WO2019/125151 describes a process where the carbon dioxide and the epoxide compound react in suspension of liquid cyclic carbonate and a supported dimeric aluminium salen complex. According to this publication the liquid cyclic carbonate product acts as an efficient heat transfer medium which avoids overheating. In this process the deactivated dimeric aluminium salen complex is reactivated by contacting the complex with a halide compound acting as an activating compound. The reactivation may be by adding the halide compound to the deactivated dimeric aluminium salen complex in a separate step while not adding extra carbon dioxide and epoxide compound. The reaction may be performed in a series of continuously stirred reactors wherein in the last reactor the cyclic carbonate product is separated from the supported dimeric aluminium salen complex. A problem with this type of reactor configuration is that lower conversions are obtained and higher reactor volumes are required to achieve the desired production capacity. Additionally, operational issues like clogging, blockage, pump failure, erosion, wear, tear and leakages can occur with this type of reactor configuration.

The object of this invention is to provide a process which does not have the disadvantages as described for the process of WO2019/125151. This is achieved with the following process.

Process to continuously prepare a cyclic carbonate product by reacting an epoxide compound with carbon dioxide in the presence of a heterogeneous catalyst which catalyst is activated by an activating compound,

wherein the process is performed in at least a first, second, third reactor, each reactor comprising a slurry of the heterogeneous catalyst and the cyclic carbonate product as present as a liquid,

wherein to the first reactor carbon dioxide and the epoxide compound is continuously supplied, liquid cyclic carbonate is discharged as a first product stream and unreacted carbon dioxide and epoxide are discharged as a first gaseous effluent stream while substantially all of the heterogeneous catalyst remains in the first reactor and wherein the heterogeneous catalyst deactivates in time,

wherein to the second reactor the first gaseous effluent is continuously supplied, liquid cyclic carbonate is discharged as a second product stream and unreacted carbon dioxide and epoxide is discharged as a second gaseous effluent stream while substantially all of the heterogeneous catalyst remains in the second reactor,

wherein to the third reactor the activating compound is added to activate the heterogeneous catalyst thereby obtaining a reactor comprising activated heterogeneous catalyst and

wherein in a next step of the process the third reactor becomes the second reactor, the second reactor becomes the first reactor and the first reactor becomes the third reactor such to activate the deactivated catalyst present in said reactor.

Applicants found that by performing the process according to the invention a more efficient conversion of the epoxide compound to the desired cyclic carbonate product is possible. Further no catalyst containing suspension has to be moved, ie pumped, from one reactor to another reactor because substantially all of the heterogeneous catalyst remains in the same reactor.

In first and second reactor the carbon dioxide is contacted with the epoxide compound in a suspension of liquid cyclic carbonate. The temperature and pressure conditions are chosen such that the cyclic carbonate is in its liquid state. The temperature and pressure conditions are further chosen such that carbon dioxide and epoxide easily dissolve in the liquid cyclic carbonate reaction medium. The temperature may be between 0 and 200° C. and the pressure is between 0 and 5.0 MPa (absolute) and wherein temperature is below the boiling temperature of the cyclic carbonate product at the chosen pressure. At the high end of these temperature and pressure ranges complex reactor vessels will be required. Because favourable results with respect to selectivity and yield to the desired carbonate product are achievable at lower temperatures and pressures it is preferred that the temperature in the first and second reactor is between 20 and 150° C., more preferably between 40 and 120° C., and the absolute pressure is between 0.1 and 0.5 MPa, more preferably between 0.1 and 0.3 MPa. The pressure in the first reactor may be higher than the pressure in the second reactor. This is advantageous because no special measures, such as compressors or blowers, have to be present to create a flow of the first gaseous effluent to the second reactor.

In the process according to the invention the first, second and third reactor change their relative operating mode after each step of the process. One step of the process involves operating the first and second reactor as described to prepare the cyclic carbonate product while the catalyst in the third reactor is regenerated. In the first reactor a bulk operation takes place, in the second reactor a polishing operation takes place and in the third reactor a regeneration operation takes place. One physical reactor may thus be the first, second and third reactor in different steps of the process. At the start of a next step the third reactor, ie the reactor which has just been regenerated, becomes the second reactor, ie the polishing reactor, the second reactor becomes the first reactor, ie the bulk reactor and the first reactor becomes the third reactor such that the deactivated catalyst is regenerated. This may be achieved by operating a set of sequence valves which result in that the previous third reactor is connected to the previous second reactor in such a way that these reactors will operate as the first and second reactor according to the invention. The previous first reactor, comprising deactivated heterogeneous catalyst, is disconnected from the supply conduits for carbon dioxide and epoxide compound and connected to a supply conduit for the activating compound. The time period of one step may be between 1-30 days, preferably between 2-20 days. In such a period of time cyclic carbonate product may be continuously be prepared in the first and second reactor. The addition of the activating compound to the third reactor to obtain a reactor comprising activated heterogeneous catalyst may be performed in a shorter time period.

Regeneration of the catalyst in the third reactor by adding the activating compound is thus performed in a so-called off-line mode where no reactants, such as carbon dioxide and epoxide or effluents from the first or the second reactor are supplied to the third reactor.

The process may be performed in more than the 3 reactors specified above, referred to as a reactor train. For example more than one reactor train may be operated in parallel according to the process of this invention. The reactor effluents of these trains may be separated into products and activating compounds in a common separation process.

A reactor train may comprise a further reactor, referenced as the intermediate reactor. The intermediate reactor comprises a slurry of the heterogeneous catalyst and the cyclic carbonate product as present as a liquid similar to the other reactors. To the intermediate reactor the gaseous effluent of an upstream reactor in the reactor train is continuously supplied, liquid cyclic carbonate is discharged as an intermediate reactor product stream and unreacted carbon dioxide and epoxide is discharged as an intermediate reactor gaseous effluent stream. Substantially all of the heterogeneous catalyst remains in the intermediate reactor. A train with more than 3 reactors will comprise of the first, second and third reactor according to the process of the invention. The additional reactors will operate in series with the first and second reactor, wherein the additional reactors will be placed between first and second reactor.

The reactors may be any reactor in which the reactants and catalyst in the liquid reaction mixture can intimately contact and wherein the feedstock can be easily supplied to. The reactor is a continuously operated reactor when used as first and second reactor. To such a reactor carbon dioxide and the epoxide compound is continuously supplied and liquid cyclic carbonate is discharged. The speed at which the gaseous carbon dioxide and the gaseous or liquid epoxide is supplied could agitate the liquid contents of the reactor such that a substantially evenly distributed reaction mixture results. Sparger nozzle may be used to add a gaseous compound to the reactor. Such agitation may also be achieved by using for example ejectors or mechanical stirring means, like for example impellers. Such reactors may be of the so-called bubble column slurry type reactor and mechanically agitated stirred tank reactor. In a preferred embodiment the reactor is a continuously operated stirred reactor wherein carbon dioxide and epoxide compound are continuously supplied to the reactor and wherein part of the cyclic carbonate product is continuously withdrawn as part of a liquid stream. The reactors of a reactor train are preferably of the same size and design. The reactors of parallel operated reactor trains may be different for each train.

In the process of the invention substantially all of the heterogeneous catalyst remains in the reactor while part of the liquid cyclic carbonate product is discharged from the reactor. Preferably a volume of liquid cyclic carbonate product is discharged from the first reactor and second reactor and any optional intermediate reactor(s) which corresponds with the production of cyclic carbonate product in the reactor such that the volume of suspension in the reactor remains substantially the same during the step. The liquid cyclic carbonate is separated from the heterogeneous catalyst by a filter. This filter may be positioned external of the reactor. Preferably the filter is positioned within the reactor. A vertical positioned cylindrical vessel is preferred. A preferred filter is a cross-flow filter. For the preferred supported dimeric aluminium salen complex as the catalyst is a 10 μm filter, more preferably composed of a so-called Johnson Screens® using Vee-Wire® filter elements, is preferred. The filter may have the shape of a tube placed vertically in the reactor, preferably in the preferred vertical positioned cylindrical vessel. The filter may be provided with means to create a negative flow over the filter such to remove any solids from the filter opening.

Suitably part of the second gaseous effluent is recycled to the first reactor and part of the second gaseous effluent is purged from the process. Because content of non-reacted epoxide in the second gaseous effluent is minimised as a result of the reactor layout according to the process of this invention the loss of epoxide via the purge is also minimised.

The first product stream and the second product stream and any optional intermediate reactor product stream may still comprise some epoxide compound and some activator compound. This epoxide compound is suitably separated from the product stream and returned to any one of the first, second or optional intermediate reactors. More preferably the first product stream and the second product stream and any optional intermediate reactor product streams are contacted with carbon dioxide resulting in a cleaned product stream and a loaded carbon dioxide stream containing epoxide compound. More preferably the first product stream and the second product stream are combined in a combined stream and wherein epoxide present in the combined product stream is stripped out by contacting the combined product stream with carbon dioxide resulting in a cleaned product stream and a loaded carbon dioxide stream containing epoxide compound and wherein the loaded carbon dioxide stream is supplied to the first reactor.

The cleaned product stream will still contain some activator compound, carbon dioxide and epoxide compound. The content of activator compound in this stream may vary over time. For example at the start of a step the content of activator compound may be high due to the fact that a freshly regenerated reactor is put on stream. During the step the content of activator compound will gradually decrease. Suitably the cyclic carbonate product as present in cleaned product stream is separated from the activating compound as present in the combined product stream in a distillation step wherein a purified cyclic carbonate product is obtained as a bottom product of the distillation step. The activating compound obtained as the top product in this distillation may be further purified by separation of any entrained gasses, such as carbon dioxide and epoxide compound. The activating compound obtained in the distillation step may be used to activate the deactivated catalyst in the third reactor. The activating compound is suitably directly added to the third reactor and/or stored. The stored activator compound may then be added at another moment in time to the third reactor.

Applicants found that the distillation during a step was difficult to perform due to variances in flows and concentrations and found that when the first product stream and the second product stream and/or the combined stream pass a buffer vessel upstream of the distillation step a more stable distillation may be performed. When the heterogeneous catalyst is a preferred supported dimeric aluminium salen complex and the activating compound is a halide compound it is preferred that the volume of the buffer vessel or vessels expressed in m³ relative to the amount of dimeric aluminium salen complex as present in the reactors in which the reaction to the cyclic carbonate product takes place, ie the first and second reactor and expressed in kmol is between 5 and 50 m3/kmol.

Preferably all or part of the epoxide as obtained in the distillation is directly or indirectly recycled to the first reactor. In this way all or almost all of the epoxide can be converted to the cyclic carbonate product. Part of the epoxide as obtained in the distillation may be purged such to avoid a build-up of compounds boiling in the same range as the epoxide. These other compounds may have been present in any one of the feedstocks or which may have formed in the process. The heterogeneous catalyst may be any catalyst suited to catalyse the reaction of carbon dioxide and an epoxide to a cyclic carbonate and which is suitably activated by a halide compound. More especially heterogeneous catalyst comprising an organic compound containing one or more nucleophilic groups such as quaternary nitrogen halides. Examples of such catalysts are described in

Kohrt, C.; Werner, T. Recyclable Bifunctional Polystyrene and Silica Gel-Supported Organocatalyst for the Coupling of CO2 with Epoxides. ChemSusChem 2015, 8, 2031-2034,

Zakharova, M.V.; Kleitz, F.; Fontaine, F.-G. Carbon Dioxide Oversolubility in Nanoconfined Liquids for the Synthesis of Cyclic Carbonates. ChemCatChem 2017, 9, 1886-1890,

Kolle, J.M.; Sayari, A. Substrate dependence on the fixation of CO2 to cyclic carbonates over reusable porous hybrid solids. J. CO2 Util. 2018, 26, 564-574,

Liu, M.; Liu, B.; Liang, L.; Wang, F.; Shi, L.; Sun, J. Design of bifunctional NH3l-Zn/SBA-15 single-component heterogeneous catalyst for chemical fixation of carbon dioxide to cyclic carbonates. J. Mol. Catal. A Chem. 2016, 418-419, 78-85, and

Lan, D.-H.; Chen, L.; Au, C.-T.; Yin, S.-F. One-pot synthesized multi-functional graphene oxide as a water-tolerant and efficient metal-free heterogeneous catalyst for cycloaddition reaction. Carbon 2015, 93, 22-31;

DABCO-based ammonium salts as described in Hajipour, A.R.; Heidari, Y.; Kozehgary, G. Silica grafted ammonium salts based on DABCO as heterogeneous catalysts for cyclic carbonate synthesis from carbon dioxide and epoxides. RSC Adv. 2015, 5, 22373-22379;

Bifunctional resorcinarenes salts, with ammonium-groups, as described in Jose, T., Canellas, S., Pericas, M.A., Kleijn, A.W., Polystyrene-supported bifunctional resorcinarenes as cheap, metal-free and recycle catalyst for epoxide/CO2 coupling reactions, Green Chemistry 2017, 19, 5488;

Imidazolium-based ionic liquids and salts as described in any one of the following papers:

Xiao, L.-F.; Li, F.-W.; Peng, J.-J.; Xia, C.-G. Immobilized ionic liquid/zinc chloride: Heterogeneous catalyst for synthesis of cyclic carbonates from carbon dioxide and epoxides. J. Mol. Catal. A Chem. 2006, 253, 265-269,

Han, L.; Park, S.-W.; Park, D.-W. Silica grafted imidazolium-based ionic liquids: Efficient heterogeneous catalysts for chemical fixation of CO2 to a cyclic carbonate. Energy Environ. Sci. 2009, 2, 1286-1292,

Sadeghzadeh, S.M. A heteropolyacid-based ionic liquid immobilized onto fibrous nano-silica as an efficient catalyst for the synthesis of cyclic carbonate from carbon dioxide and epoxides. Green Chem. 2015, 17, 3059-3066,

Liu, M.; Lu, X.; Jiang, Y.; Sun, J.; Arai, M. Zwitterionic Imidazole-Urea Derivative Framework Bridged Mesoporous Hybrid Silica: A Highly Efficient Heterogeneous Nanocatalyst for Carbon Dioxide Conversion. ChemCatChem 2018, 10, 1860-1868,

Comés, A.; Collard, X.; Fusaro, L.; Atzori, L.; Cutrufello, M.G.; Aprile, C. Bi-functional heterogeneous catalysts for carbon dioxide conversion: Enhanced performances at low temperature. RSC Adv. 2018, 8, 25342-25350,

Aprile, C.; Giacalone, F.; Agrigento, P.; Liotta, L.F.; Martens, J.A.; Pescarmona, P.P.; Gruttadauria, M. Multilayered Supported Ionic Liquids as Catalysts for Chemical Fixation of Carbon Dioxide: A High-Throughput Study in Supercritical Conditions. ChemSusChem 2011, 4, 1830-1837,

Agrigento, P.; Al-Amsyar, S.M.; Sorée, B.; Taherimehr, M.; Gruttadauria, M.; Aprile, C.; Pescarmona, P.P. Synthesis and high-throughput testing of multilayered supported ionic liquid catalysts for the conversion of CO2 and epoxides into cyclic carbonates. Catal. Sci. Technol. 2014, 4, 1598-1607,

Calabrese, C.; Liotta, L.F.; Giacalone, F.; Gruttadauria, M.; Aprile, C. Supported Polyhedral Oligomeric Silsesquioxane-Based (POSS) Materials as Highly Active Organocatalysts for the Conversion of CO2. ChemCatChem 2019, 11, 560-567,

Lee, J.H.; Lee, A.S.; Lee, J.-C.; Hong, S.M.; Hwang, S.S.; Koo, C.M. Multifunctional Mesoporous Ionic Gels and Scaffolds Derived from Polyhedral Oligomeric Silsesquioxanes. ACS Appl. Mater. Interfaces 2017, 9, 3616-3623,

Akbari, Z.; Ghiaci, M. Heterogenization of a Green Homogeneous Catalyst: Synthesis and Characterization of Imidazolium lonene/Br—Cl—@SiO2 as an Efficient Catalyst for the Cycloaddition of CO2 with Epoxides. Ind. Eng. Chem. Res. 2017, 56, 9045-9053,

Su, Q.; Qi, Y.; Yao, X.; Cheng, W.; Dong, L.; Chen, S.; Zhang, S. Ionic liquids tailored and confined by one-step assembly with mesoporous silica for boosting the catalytic conversion of CO2 into cyclic carbonates. Green Chem. 2018, 20, 3232-3241,

Han, L.; Li, H.; Choi, S.-J.; Park, M.-S.; Lee, S.-M.; Kim, Y.-J.; Park, D.-W. Ionic liquids grafted on carbon nanotubes as highly efficient heterogeneous catalysts for the synthesis of cyclic carbonates. Appl. Catal. A 2012, 429-430, 67-72,

Buaki-Sogó, M.; Vivian, A.; Bivona, L.A.; García, H.; Gruttadauria, M.; Aprile, C. Imidazolium functionalized carbon nanotubes for the synthesis of cyclic carbonates: Reducing the gap between homogeneous and heterogeneous catalysis. Catal. Sci. Technol. 2016, 6, 8418-8427,

Jayakumar, S.; Li, H.; Chen, J.; Yang, Q. Cationic Zn—Porphyrin Polymer Coated onto CNTs as a Cooperative Catalyst for the Synthesis of Cyclic Carbonates. ACS Appl. Mater. Interfaces 2018, 10, 2546-2555,

Xu, J.; Xu, M.; Wu, J.; Wu, H.; Zhang, W.-H.; Li, Y.-X. Grapheneoxide immobilized with ionic liquids: Facile preparation and efficient catalysis for solvent-free cycloaddition of CO2 to propylene carbonate. RSC Adv. 2015, 5, 72361-72368, and

Calabrese,C.;Liotta,L.F.;Carbonell,E;Giacalone,F.;Gruttadauria,M.;Aprile,C.Imidazolium -Functionalized Carbon Nanohorns for the Conversion of Carbon Dioxide: Unprecedented Increase of Catalytic Activity after Recycling. ChemSusChem 2017, 10, 1202-1209;

Phosphonium salts as described in Takahashi, T.; Watahiki, T.; Kitazume, S.; Yasuda, H.; Sakakura, T. Synergistic hybrid catalyst for cyclic carbonate synthesis: Remarkable acceleration caused by immobilization of homogeneous catalyst on silica. Chem. Commun. 2006, 1664-1666 and in Sakai, T.; Tsutsumi, Y.; Ema, T. Highly active and robust organic-inorganic hybrid catalyst for the synthesis of cyclic carbonates from carbon dioxide and epoxides. Green Chem. 2008, 10, 337-341;

Pyridinium salts as described in Motokura, K.; Itagaki, S.; Iwasawa, Y.; Miyaji, A.; Baba, T. Silica-supported aminopyridinium halides for catalytic transformations of epoxides to cyclic carbonates under atmospheric pressure of carbon dioxide. Green Chem. 2009, 11, 1876-1880;

Porphyrin complexes as described in Jayakumar, S.; Li, H.; Tao, L.; Li, C.; Liu, L.; Chen, J.; Yang, Q. Cationic Zn-Porphyrin Immobilized in Mesoporous Silicas as Bifunctional Catalyst for CO2 Cycloaddition Reaction under Cocatalyst Free Conditions. ACS Sustain. Chem. Eng. 2018, 6, 9237-9245; and

Hybrid Amine-Functionalized Graphene Oxide as described in Saptal, V.B.; Sasaki, T.; Harada, K.; Nishio-Hamane, D.; Bhanage, B.M. Hybrid Amine-Functionalized Graphene Oxide as a Robust Bifunctional Catalyst for Atmospheric Pressure Fixation of Carbon Dioxide using Cyclic Carbonates. ChemSusChem 2016, 9, 644-650.

A preferred heterogeneous catalyst is a supported dimeric aluminium salen complex and the activating compound is a halide compound .

The supported dimeric aluminium salen complex may be any supported complex as disclosed by the earlier referred to EP2257559B1. Preferably the complex is represented by the following formula:

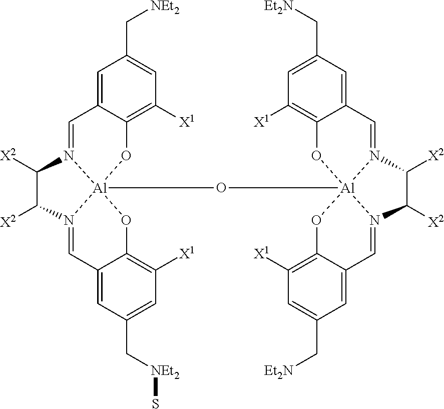

wherein S represents a solid support connected to the nitrogen atom via an alkylene bridging group, wherein the supported dimeric aluminium salen complex is activated by a halide compound. The alkylene bridging group may have between 1 and 5 carbon atoms. X² may be a C6 cyclic alkylene or benzylene. Preferably X² is hydrogen. X¹ is preferably a tertiary butyl. Et in the above formula represents any alkyl group, preferably having from 1 to 10 carbon atoms. Preferably Et is an ethyl group.

S represents a solid support. The catalyst complex may be connected to such a solid support by (a) covalent binding, (b) steric trapping or (c) electrostatic binding. For covalent binding, the solid support S needs to contain or be derivatized to contain reactive functionalities which can serve for covalently linking a compound to the surface thereof.

Such materials are well known in the art and include, by way of example, silicon dioxide supports containing reactive Si-OH groups, polyacrylamide supports, polystyrene supports, polyethyleneglycol supports, and the like. A further example is sol-gel materials. Silica can be modified to include a 3-chloropropyloxy group by treatment with (3-chloropropyl) triethoxysilane. Another example is Al pillared clay, which can also be modified to include a 3-chloropropyloxy group by treatment with (3-chloropropyl)triethoxysilane. Solid supports for covalent binding of particular interest in the present invention include siliceous MCM-41 and MCM-48, optionally modified with 3-aminopropyl groups, ITQ-2 and amorphous silica, SBA-15 and hexagonal mesoporous silica. Also of particular interest are sol-gels. Other conventional forms may also be used. For steric trapping, the most suitable class of solid support is zeolites, which may be natural or modified. The pore size must be sufficiently small to trap the catalyst but sufficiently large to allow the passage of reactants and products to and from the catalyst. Suitable zeolites include zeolites X, Y and EMT as well as those which have been partially degraded to provide mesopores, that allow easier transport of reactants and products. For the electrostatic binding of the catalyst to a solid support, typical solid supports may include silica, Indian clay, Al-pillared clay, Al-MCM-41, K10, laponite, bentonite, and zinc-aluminium layered double hydroxide. Of these silica and montmorillonite clay are of particular interest. Preferably the support S is a particle chosen from the group consisting of silica, alumina, titania, siliceous MCM-41 or siliceous MCM-48.

Preferably the support S has the shape of a powder having dimensions which are small enough to create a high active catalytic surface per weight of the support and large enough to be easily separated from the cyclic carbonate in or external of the reactor. Preferably the support powder particles have for at least 90 wt % of the total particles a particle size of above 10 μm and below 2000 μm. The particle size is measured by a Malvern® Mastersizer® 2000.

The supported catalyst complex as shown above is activated by a halide compound. The halide compound will comprise a halogen atom which halogen atom may be Cl, Br or I and preferably Br. The quaternary nitrogen atom of the complex shown above is paired with the halide counterion. Possible activating compounds are described in EP2257559B1 which exemplifies tetrabutylammonium bromide as a possible activating compound. Benzyl bromide is a preferred activating compound because it can be separated from the preferred cyclic carbonate product, such as propylene carbonate and ethylene carbonate by distillation.

An example of a preferred supported dimeric aluminium salen complex which complex is activated by benzyl bromide is shown below, wherein Et is ethyl and tBu is tert-butyl and Osilica represents a silica support:

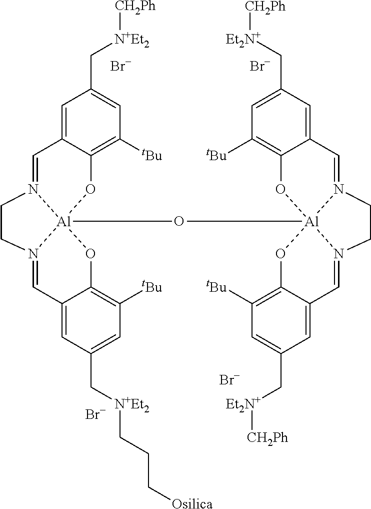

In use the Et group in the above formula may be exchanged with the organic group of the halide compound. For example if benzyl bromide is used as the halide compound to activate the above supported dimeric aluminium salen complex the Et group will be exchanged with the benzyl group when the catalyst is reactivated.

An alternative for the supported dimeric aluminium salen complex as described above may be a supported catalyst wherein an aluminium salen complex part is connected to a support. By positioning these monomers close enough to each other the same catalytic effect as with the dimeric salen complex described above may be achieved. Optionally the supported monomer aluminium salen complex may react with a neighbouring monomer aluminium salen complex to obtain a supported dimeric aluminium salen complex described above which has two connecting bridges to the support instead of one connecting bridge as described above.

The epoxide product may be the epoxides as described in the afore mentioned EP2257559B1 in paragraphs 22-26. Preferably the epoxide compound has 2 to 8 carbon atoms. Preferred epoxide compounds are ethylene oxide, propylene oxide, butylene oxide, pentene oxide, glycidol and styrene oxide. The cyclic carbonate products which may be prepared from these preferred epoxides have the general formula:

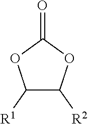

where R¹ is a hydrogen or a group having 1-6 carbon atoms, preferably hydrogen, methyl, ethyl, propyl, hydroxymethyl and phenyl, and R² is hydrogen.

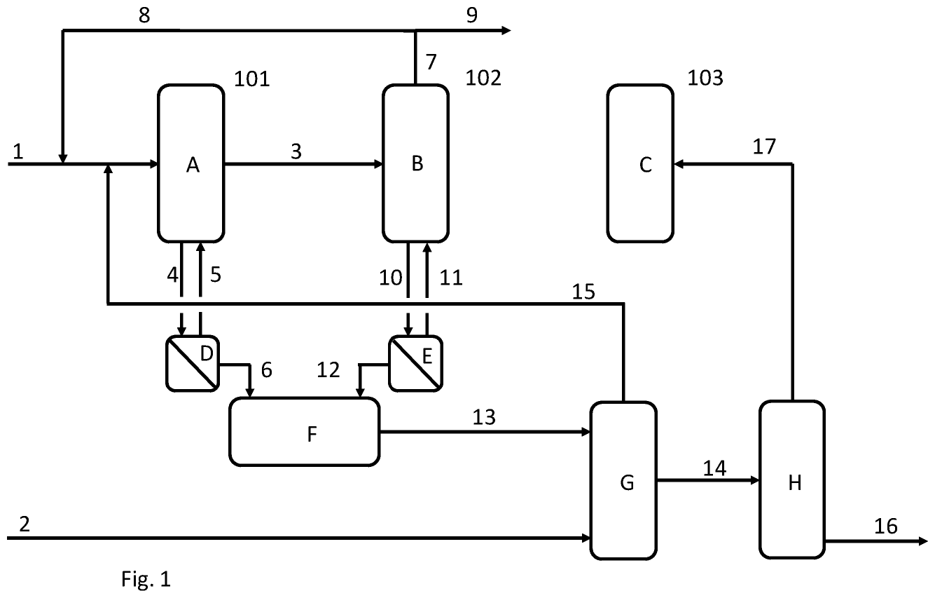

The invention shall be illustrated by FIG. 1. FIG. 1 shows a flow scheme of the process according to the invention starting from propylene oxide and using a supported dimeric aluminium salen complex as activated by benzyl bromide as the catalyst. A reactor (A) as first reactor, a reactor (B) as second reactor and a reactor (C) as third reactor is shown. All three reactors comprise of a slurry of the catalyst and the cyclic carbonate product. To reactor (A) carbon dioxide is continuously supplied via stream (2), stripper (G) and stream (15). In stripper (G) the carbon dioxide gas contacts a combined liquid product stream (13) to obtain a cleaned liquid product stream (14) and a loaded carbon dioxide stream (15) containing some propylene oxide compound. This stream (15) is combined with fresh propylene oxide as supplied via (1) and part (8) of the unreacted carbon dioxide and propylene oxide from reactor (B) as the second reactor, and the combined stream is supplied to reactor (A) which is in a bulk operation (101) as the first reactor. In reactor (A) a liquid cyclic carbonate is formed by reaction of carbon dioxide and propylene oxide. During the process step part of the slurry is discharged as stream (4) to a filter (D). In this filter liquid cyclic carbonate is separated from the catalyst. The catalyst is returned to reactor (A) via stream (5) and liquid cyclic carbonate poor in catalyst discharged as a first product stream (6) from reactor (A). Unreacted carbon dioxide and propylene oxide is discharged as a first gaseous effluent stream (3) and continuously supplied to second reactor (B) which is in a polishing operation (102).

In reactor (B) a liquid cyclic carbonate is formed by reaction of carbon dioxide and propylene oxide. During the process step part of the slurry is discharged as stream (10) to a filter (E). In this filter liquid cyclic carbonate is separated from the catalyst. The catalyst is returned to reactor (B) via stream (11) and liquid cyclic carbonate poor in catalyst discharged as a second product stream (12) from reactor (B). Unreacted carbon dioxide and propylene oxide is discharged as a second gaseous effluent stream (7) of which part is recycled to first reactor (A) and part is purged via stream (9).

Cleaned product streams (6) and (12) are collected in a buffer vessel (F). From this buffer vessel (F) a combined product stream (13) is fed to the stripper (G). The cleaned liquid product stream (14) is fed to a distillation column (H) wherein the cyclic carbonate product as present in cleaned product stream is separated from the benzyl bromide and other lower boiling compounds. The benzyl bromide is fed via stream (17) to the reactor (C), optionally via a storage vessel (not shown). A purified cyclic carbonate product is obtained as a bottom product (16) in the distillation column (H).

In the process step the deactivated supported dimeric aluminium salen complex as present in reactor (C) as the third reactor is activated by adding benzyl bromide via stream (17). In FIG. 1 reactor (C) is in a regeneration operation (103).

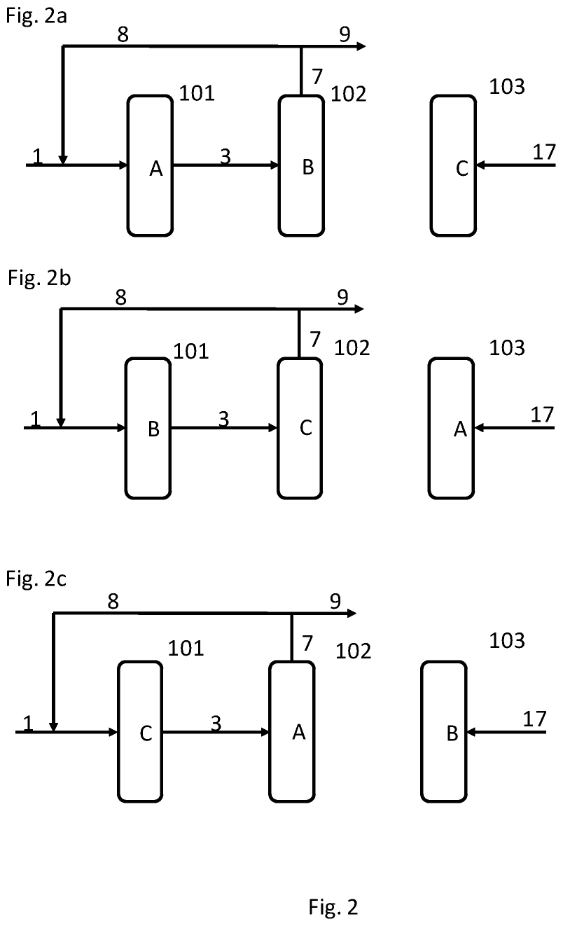

FIG. 2a shows part of the process scheme of FIG. 1. FIG. 2b shows how the physical reactors (A),(B),(C) are configured in the line-up in a next step and FIG. 2c shows how the physical reactors (A),(B),(C) are configured in the line-up in a further step.

The process step of FIG. 1 and FIG. 2a ends when the catalyst as present in reactor (C) is re-activated and/or when the catalyst activity in the combined reactor (A) and reactor (B) drops below an unacceptable level. A next step starts by switching the reactors such that the reactor (C) becomes a second reactor, , reactor (B) becomes the first reactorand reactor (A) is disconnected from the running process to be regenerated in a regeneration operation (103) as the third reactor as shown in FIG. 2b. In this way the deactivated catalyst of reactor (A) at the end of the previous step can be activated. This is repeated in a third step where the reactors (A),(B) and (C) are line up as shown in FIG. 2c. In a further next step the reactors return to their original line up of FIG. 2a. This sequence of steps is also shown in the below table 1: In this manner the catalyst as present in reactors (A), (B) and (C) remain in their physical reactor vessels while the operation performed by or in these reactors, ie bulk, polishing and regeneration, change after every step.

TABLE 1

| | | Bulk | Polishing | Regeneration |
|---|---|---|---|---|
| step | FIG. | operation (101) | operation (102) | operation (103) |
| 1 | 1, 2a | Reactor A | Reactor B | Reactor C |
| 2 | 2b | Reactor B | Reactor C | Reactor A |
| 3 | 2c | Reactor C | Reactor A | Reactor B |

The invention will be illustrated by the following non-limiting example.

### Comparative Example A

The operation as illustrated in FIGS. 1-2 is compared to an operation having a sequence of steps as shown in Table 2, wherein the reactor in bulk operation is used as the reactor in polishing operation in a next step and wherein the reactor in polishing operation is regenerated in this next step and wherein the cyclic carbonate production is equal to the operation as illustrated in FIGS. 1-2.

TABLE 2

| | Bulk | Polishing | Regeneration |
|---|---|---|---|
| step | operation (101) | operation (102) | operation (103) |
| 1 | Reactor A | Reactor B | Reactor C |
| 2 | Reactor C | Reactor A | Reactor B |
| 3 | Reactor B | Reactor C | Reactor A |

In this comparison use is made of the catalyst deactivation properties and catalyst life time properties as experimentally obtained. Catalyst deactivation is determined by the loss of activating compound and takes place on a time scale of one process step which may be between 2 and 20 days. Catalyst life time is when the moment when the catalyst after regeneration is not capable to achieve a certain desired conversion in the illustrated process line up. Catalyst life time is on a time scale of several months. The comparison with the process according to the invention is shown in Table 3.

### Comparative Example B

The operation as illustrated in FIGS. 1-2 is compared to an operation using two reactors, each having twice the volume of reactor A and containing twice the amount of catalyst. In one reactor cyclic carbonate is prepared and the other reactor is in regeneration operation. The calculation is based on a situation wherein the same cyclic carbonate production as in the process of FIGS. 1 and 2 is achieved.

TABLE 3

| | According to | Comparative | Comparative |
|---|---|---|---|
| | FIGS. 1 and 2 | example A | example B |
| Catalyst life time | 100 | 83.3 | 50 |
| loss of epoxide via | 100 | 118 | 95 |
| purge | | | |

From Table 3 it is shown that in the sequence of Example A the catalyst life time is lower and the loss of epoxide via the purge is higher than when the process is performed according to the invention. Comparative example B shows that the moment when the catalyst after regeneration is not capable to achieve a certain desired conversion (ie the catalyst life time) is significantly shorter as compared to the process according to the invention. The loss of epoxide is smaller. However this does not compensate for the fact that the catalyst will have to be changed for new catalyst at a much higher frequency when only one vessel is used as compared to the process according to the invention.

1. A process to continuously prepare a cyclic carbonate product by reacting an epoxide compound with carbon dioxide in the presence of a heterogeneous catalyst which catalyst is activated by an activating compound, wherein the process is performed in at least a first, second, third reactor, each reactor comprising a slurry of the heterogeneous catalyst and the cyclic carbonate product as present as a liquid,
wherein to the first reactor carbon dioxide and the epoxide compound is continuously supplied, liquid cyclic carbonate is discharged as a first product stream and unreacted carbon dioxide and epoxide are discharged as a first gaseous effluent stream while substantially all of the heterogeneous catalyst remains in the first reactor and wherein the heterogeneous catalyst deactivates in time,
wherein to the second reactor the first gaseous effluent is continuously supplied, liquid cyclic carbonate is discharged as a second product stream and unreacted carbon dioxide and epoxide is discharged as a second gaseous effluent stream while substantially all of the heterogeneous catalyst remains in the second reactor,
wherein to the third reactor the activating compound is added to activate the heterogeneous catalyst thereby obtaining a reactor comprising activated heterogeneous catalyst and wherein in a next step of the process the third reactor becomes the second reactor, the second reactor becomes the first reactor and the first reactor becomes the third reactor such to activate the deactivated catalyst present in said reactor.

2. The process according to claim 1, wherein the temperature in the first and second reactor is between 20 and 150° C. and the absolute pressure is between 0.1 and 0.5 MPa and wherein temperature is below the boiling temperature of the cyclic carbonate product at the chosen pressure.

3. The process according to claim 1, wherein the heterogeneous catalyst comprises an organic compound containing one or more nucleophilic groups.

4. The process according to claim 3, wherein the nucleophilic group is a quaternary nitrogen halide.

5. The process according to claim 4, wherein the heterogeneous catalyst is a supported dimeric aluminium salen complex and the activating compound is a halide compound.

6. The process according to claim 5, wherein the supported dimeric aluminium salen complex is represented by the following formula: 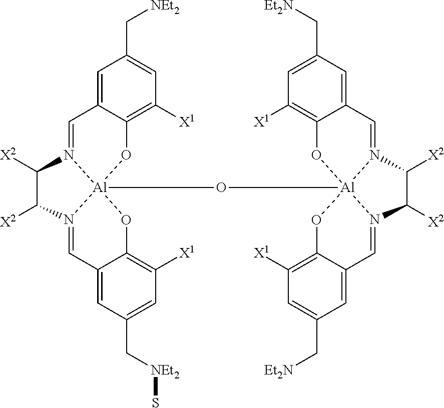
wherein S represents a solid support connected to the nitrogen atom via an alkylene group, wherein the supported dimeric aluminium salen complex is activated by a halide compound and wherein X¹ is tertiary butyl and X² is hydrogen and wherein Et is an alkyl group having 1 to 10 carbon atoms.

7. The process according to claim 6, wherein the support S is composed of particles having an average diameter of between 10 and 2000 μm.

8. The process according to claim 7, wherein the support S is a particle chosen from the group consisting of silica, alumina, titania, siliceous MCM-41 or siliceous MCM-48.

9. The process according to claim 5, wherein the halide compound is benzyl halide.

10. The process according to claim 9, wherein the benzyl halide is benzyl bromide.

11. The process according to claim 1, wherein the time period of one step is between 1-30 days.

12. The process according to claim 1, wherein the first product stream and the second product stream are combined in a combined stream and wherein epoxide present in the combined product stream is stripped out by contacting the combined product stream with carbon dioxide resulting in a cleaned product stream and a loaded carbon dioxide stream containing epoxide compound and wherein the loaded carbon dioxide stream is supplied to the first reactor.

13. The process according to claim 12, wherein the cyclic carbonate product as present in cleaned product stream is separated from the activating compound as present in the combined product stream in a distillation step wherein a purified cyclic carbonate product is obtained as a bottom product of the distillation step.

14. The process according to claim 13, wherein the activating compound obtained in the distillation step is used to activate the deactivated catalyst in the third reactor.

15. The process according to claim 13, wherein the first product stream and the second product stream and/or the combined stream pass a buffer vessel upstream of the distillation step.

16. The process according to claim 15, wherein the heterogeneous catalyst is a supported dimeric aluminium salen complex and the activating compound is a halide compound. and wherein the volume of the buffer vessel or vessels expressed in m³ relative to the amount of dimeric aluminium salen complex as present in the first and second reactor and expressed in kmol is between 5 and 50 m³/kmol.

17. The process according to claim 1, wherein part of the second gaseous effluent is recycled to the first reactor and part of the second gaseous effluent is purged from the process.

18. The process according to claim 1, wherein the epoxide compound has 2 to 8 carbon atoms.

19. The process according to claim 18, wherein the epoxide compound is ethylene oxide, propylene oxide, butylene oxide, pentene oxide, glycidol or styrene oxide.
